SEQUENCE LISTING (7 sequences)

SEQ ID NO: 1 - protein, 13 aa, Microciona prolifera
GVSVRRYRNRVRI

SEQ ID NO: 2 - protein, 15 aa, Artificial
  artificial peptide derived from Microciona prolifera
VGFDPYDYEVNEADG

SEQ ID NO: 3 - protein, 10 aa, Artificial
  artificial peptide derived from Microciona prolifera
EDQLDAMNLS

SEQ ID NO: 4 - protein, 11 aa, Artificial
  artificial peptide derived from Microciona prolifera
GSGIGDEPTTS

SEQ ID NO: 5 - protein, 12 aa, Artificial
  artificial peptide derived from Microciona prolifera
RFVINITTSGSD

SEQ ID NO: 6 - protein, 12 aa, Artificial
  artificial peptide derived from Microciona prolifera
CFLTPHGVELHK

SEQ ID NO: 7 - protein, 21 aa, Artificial
  artificial peptide derived from Microciona prolifera
NGSIGPRGLPGVRGDRGKRGK

(19) United States
(12) Patent Application Publication
(10) Pub. No.: US 2004/0259783 A1
(43) Pub. Date: Dec. 23, 2004

(54) Pharmaceutical compositions of marine sponge microciona prolifera

(76) Inventors: Tony Cruz, Toronto (CA); Aleksandra Pastrak, Toronto (CA); William Kuhns, Toronto (CA)

Correspondence Address: FISH & RICHARDSON P.C., 3300 DAIN RAUSCHER PLAZA, MINNEAPOLIS, MN, 55402, US

(21) Appl. No.: 10/494,859

(22) PCT Filed: Nov. 8, 2002
(86) PCT No.: PCT/CA02/01735

Related U.S. Application Data
(60) Provisional application No. 60/337,740, filed on Nov. 9, 2001.

(51) Int. Cl. A61K 38/17; C07K 14/47

(52) U.S. Cl. 514/12; 530/350

(57) ABSTRACT

The present invention provides compositions and methods for treating restenosis and autoimmune diseases. The methods provided herein include the administration of a composition comprising of the class of polypeptides, carbohydrates and/or extracts and their analogues, derivatives and conjugates, originating from the marine sponge Microciona prolifera.

### FIELD OF THE INVENTION

The present invention provides pharmaceutical compositions and methods of use for the treatment of disorders with compounds originating from the marine sponge Microciona prolifera.

### BACKGROUND OF THE INVENTION

Vascular, proliferative, autoimmune, and viral diseases are widespread, devastating diseases that affect millions of people worldwide. Such diseases include myasthenia gravis, systemic lupus erythematosus, atopic dermatitis, idiopathic pulmonary fibrosis, multiple sclerosis, diabetes, diabetic neuropathy, arthritis and other inflammatory joint diseases, cancer and metastasis, Alzheimer's disease, Parkinson's disease, amitrophic lateral sclerosis, obesity, osteoporosis, inflammatory dermatosis, inflammatory bowel disease, restenosis following vascular and coronary intervention, arteriosclerosis, wound healing disorders, hepatitis B, hepatitis C, HIV, Herpes simplex virus, genital warts, respiratory syncytial virus, rhinoviruses, adenoviruses, influenza and parainfluenza viruses and corona viruses.

Currently, compounds for the treatment of these diseases are aimed towards altering the immune system, cell proliferation, cell adhesion and migration, and cytokine levels or activities.

There are several classes of molecules and disease processes that are common to all these diseases. These include increased expression of adhesion molecules, cytokines and matrix metalloproteinases, increased cell proliferation and migration, increased inflammatory cell activation and infiltration, increased angiogenesis, and increased tissue destruction and dysfunctional matrix remodeling. Experimental and clinical studies show that tissue injury leads to the transition of cells to an activated state, followed by their proliferation and migration. Cellular migration and proliferation are facilitated by the release of matrix metalloproteinases that degrade the extracellular matrix. Certain components, such as hyaluronic acid, are especially detrimental since it functions as a cellular signaling molecule, eliciting the transition of cells to an activated state, with progression along pathways leading to cell proliferation and migration.

Sponges, the simplest multicellular organisms, are thought to have evolved from their unicellular ancestors by developing cell-recognition and adhesion mechanisms to discriminate against “non-self.” Cellular adhesion of marine sponges is an event that involves adherence of extracellular proteoglycan-like molecules, otherwise known as aggregation factors (Jarchow et al., 2000).

The protein and carbohydrate components of sponge aggregation factors assemble to form a supramolecular complex very similar to classical proteoglycans. Although the role of many glycans in nature remains unclear, they have been implicated in many biological processes, including correct folding and secretion of proteins, and as receptor sites for various microorganisms and viruses (Spillmann et al., 1995)

The aggregation factor of Microciona sponge (MAF) is an adhesive proteoglycan-like molecule with a high carbohydrate content and a protein core. Fernandez-Busquets et al. have shown that a 35 kDa protein is the basic unit of the core (MAFp3) with a binding sequence bearing a similarity to some other HA binding proteins (Fernandez-Busquets et al., 1996).

There is growing evidence that carbohydrates, found on the surfaces of all living cells, are functional constituents in cell-cell interactions. Cell-cell interactions play an important role in the development, maintenance, and pathogenesis of tissues. They are highly dynamic processes that include migration, recognition, signaling, adhesion, and finally attachment. Cells on their pathway to a final location have to pass and interact with their substratum formed of matrix and cell layers. Carbohydrates can, however, also lead to diseases when they are misused in pathological situations, by microorganisms or malignant cells, for instance (Heseley et al., 2001; Misevic et al., 1887).

Carbohydrates, which are the most prominent surface-exposed structures, must play an important role as recognition molecules in such processes. The rich variability of carbohydrate sequences that are presented by cell surface creates a refined pattern of potential attachment sites. Additionally, more interaction possibilities are created as carbohydrates can be branched while proteins cannot and oligosaccharide chains can be attached to the protein backbone in different densities and patterns.

Although the compounds for the treatment of the above listed diseases are aimed towards altering different pathological processes there is a need to develop improved therapies for restenosis, proliferative, autoimmune and viral diseases.

### SUMMARY OF THE INVENTION

Within the context of the present invention, it should be noted that the above-noted diseases are deemed to be “treated” if the typical disease course is altered, slowed, inhibited, or prevented, for at least one symptom or sign of the disease.

The present invention provides pharmaceutical compositions for treating restenosis and autoimmune diseases, methods of treating restenosis and autoimmunes disease with those pharmaceutical compositions and use of the pharmaceutical compositions to treat restenosis and autoimmune diseases.

According to an aspect of the present invention, there is provided a polypeptide comprising an amino acid sequence selected from the group consisting of B-2 (SEQ ID NO: 2), B-3 (SEQ ID NO 3), B-6 (SEQ ID NO 4), B-9 (SEQ ID NO: 5), B-10 (SEQ ID NO: 6), and B-11 (SEQ ID NO: 7).

According to another aspect of the present invention, there is provided a pharmaceutical composition comprising a polypeptide as disclosed above.

According to another aspect of the present invention, the pharmaceutical composition as described above is used for the treatment of restenosis, with or without the addition of a pharmaceutically acceptable carrier.

According to another aspect of the present invention, the pharmaceutical composition as described above is used for the treatment of an autoimmune disease, with or without the addition of a pharmaceutically acceptable carrier.

According to a further aspect of the present invention, the said autoimmune disease is selected from the group consisting of multiple sclerosis, diabetes, and an inflammatory joint disease (such as arthritis).

According to another aspect of the present invention, the pharmaceutical composition as described above is used for the treatment of multiple sclerosis.

According to yet another aspect of the present invention there is provided an antibody which binds to a polypeptide comprising an amino acid sequence selected from the group consisting of B-1 (SEQ ID NO: 1), B-2 (SEQ ID NO: 2), B-3 (SEQ ED NO 3), B-6 (SEQ ID NO 4), B-9 (SEQ ID NO: 5), B-10 (SEQ ID NO: 6), B-11 (SEQ ID NO: 7) and analogues and derivatives thereof.

According to another aspect of the present invention, said antibody is a human monoclonal antibody or an Fab fragment of an antibody.

A further aspect of the present invention is immunization therapies and vaccine compositions comprising polypeptides listed above.

According to a further aspect of the present invention there is provided a method for treating restenosis comprising the step of administering to a patient a compound selected from the group consisting of (a) a polypeptide comprising an amino acid sequence selected from the group consisting of B-1 (SEQ ID NO: 1), B-2 (SEQ ID NO: 2), B-3 (SEQ ID NO 3), B-6 (SEQ ID NO 4), B-9 (SEQ ID NO: 5), B-10 (SEQ ID NO: 6), B-11 (SEQ ID NO: 7), and analogues and derivatives thereof; and (b) an antibody to the polypeptide of (a).

According to a further aspect of the present invention there is provided a method for treating autoimmune diseases (such as multiple sclerosis, diabetes, and inflammatory joint disease (such as arthritis)) comprising the step of administering to a patient a compound selected from the group consisting of (a) a polypeptide comprising an amino acid sequence selected from the group consisting of B-1 (SEQ ID NO: 1), B-2(SEQ ID NO: 2), B-3 (SEQ ID NO 3), B-6 (SEQ ID NO 4), B-9 (SEQ ID NO: 5), B-10 (SEQ ID NO: 6), B-11 (SEQ ID NO: 7), and analogues and derivatives thereof; and (b) an antibody to the polypeptide of (a).

According to a further aspect of the present invention there is provided a method for treating multiple sclerosis comprising the step of administering to a patient a compound selected from the group consisting of (a) a polypeptide comprising an amino acid sequence selected from the group consisting of B-1 (SEQ ID NO: 1), B-2 (SEQ ID NO: 2), B-3 (SEQ ID NO 3), B-6 (SEQ ID NO 4), B-9 (SEQ ID NO: 5), B-10 (SEQ ID NO: 6), B-11 (SEQ ID NO: 7), and analogues and derivatives thereof; and (b) an antibody to the polypeptide of (a).

According to yet another aspect of the invention is a method for treating restenosis comprising the step of administering to a patient a compound selected from the group consisting of:

a. GlcNAcβ1-3Fuc;

b. Galβ1-4GlcNAcβ1-3Fuc;

c. (4,6-Pyr)Galβ1-4GlcNAcβ1-3Fuc;

d.

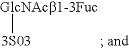

e. analogues and derivatives thereof.

According to yet another aspect of the invention is a method for treating an autoimmune disease (such as multiple sclerosis, diabetes, and inflammatory joint disease (such as arthritis)) comprising the step of administering to a patient a compound selected from the group consisting of:

a. GlcNAcβ1-3Fuc;

b. Galβ1-4GlcNAcβ1-3Fuc;

c. (4,6-Pyr)Galβ1-4GlcNAcβ1-3Fuc;

d.

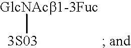

e. analogues and derivatives thereof.

According to yet another aspect of the invention is a method for treating multiple sclerosis comprising the step of administering to a patient a compound selected from the group consisting of:

a. GlcNAcβ1-3Fuc;

b. Galβ1-4GlcNAcβ1-3Fuc;

c. (4,6-Pyr)Galβ1-4GlcNAcβ1-3Fuc;

d.

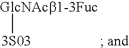

e. analogues and derivatives thereof.

According to yet another aspect of the present invention there is provided a method for treating restenosis comprising the step of administering to a patient a pharmaceutical composition comprising a purified extract from Microciona prolifera.

According to yet another aspect of the present invention there is provided a method for treating autoimmune diseases (such as multiple sclerosis, diabetes, and inflammatory joint disease (such as arthritis)) comprising the step of administering to a patient a pharmaceutical composition comprising a purified extract from Microciona prolifera.

According to yet another aspect of the present invention there is provided a method for treating multiple sclerosis comprising the step of administering to a patient a pharmaceutical composition comprising a purified extract from Microciona prolifera.

According to yet another aspect of the present invention, there is provided the method of treating restenosis comprising a step of administering to a patient at least one compound selected from a group consisting of (a) a polypeptide comprising an amino acid sequence selected from the group consisting of B-1 (SEQ ID NO: 1), B-2 (SEQ ID NO: 2), B-3 (SEQ ID NO 3), B-6 (SEQ ID NO 4), B-9 (SEQ ID NO: 5), B-10 (SEQ ID NO: 6), B-11 (SEQ ID NO: 7) and analogues and derivatives thereof; and (b) an antibody to said polypeptide.

According to yet another aspect of the present invention, there is provided the method of treating autoimmune diseases (such as multiple sclerosis, diabetes, and inflammatory joint disease (such as arthritis)) comprising a step of administering to a patient at least one compound selected from a group consisting of (a) a polypeptide comprising an amino acid sequence selected from the group consisting of B-1 (SEQ ID NO: 1), B-2 (SEQ ID NO: 2 ), B-3 (SEQ ID NO 3), B-6 (SEQ ID NO 4), B-9 (SEQ ID NO: 5), B-1 (SEQ ID NO: 6), B-11 (SEQ ID NO: 7) and analogues and derivatives thereof; and (b) an antibody to said polypeptide.

According to a further aspect of the present invention the treatment methods described above are administered wherein the dose is administered according to a regime selected from a group consisting of a single dose, multiple daily doses, multiple weekly doses and multiple monthly doses.

According to yet another aspect of the present invention, there is provided the use of polypeptide comprising an amino acid sequence selected from the group consisting of B-1 (SEQ ID NO: 1), B-2 (SEQ ID NO: 2 ), B-3 (SEQ ID NO 3), B-6 (SEQ ID NO 4), B-9 (SEQ ID NO: 5), B-10 (SEQ ID NO: 6), B-11 (SEQ ID NO: 7) and analogues and derivatives thereof for the treatment of restenosis.

According to yet another aspect of the present invention, there is provided the use of polypeptide comprising an amino acid sequence selected from the group consisting of B-1 (SEQ ID NO: 1), B-2 (SEQ ID NO: 2 ), B-3 (SEQ ID NO 3), B-6 (SEQ ID NO 4), B-9 (SEQ ID NO: 5), B-10 (SEQ ID NO: 6), B-11 (SEQ ID NO: 7) and analogues and derivatives thereof for the treatment of an autoimmune disease (such as multiple sclerosis, diabetes, and inflammatory joint disease (such as arthritis)).

According to yet another aspect of the present invention, there is provided the use of polypeptide comprising an amino acid sequence selected from the group consisting of B-1 (SEQ ID NO: 1), B-2 (SEQ ID NO: 2 ), B-3 (SEQ ID NO 3), B-6 (SEQ ID NO 4), B-9 (SEQ ID NO: 5), B-10 (SEQ ID NO: 6), B-11 (SEQ ID NO: 7) and analogues and derivatives thereof for the treatment of multiple sclerosis.

The invention further provides for the use of a purified extract from Microciona prolifera or a compound selected from the group consisting of:

a. GlcNAcβ1-3Fuc;

b. Galβ1-4GlcNAcβ1-3Fuc;

c. (4,6-Pyr)Galβ1-4GlcNAcβ1-3Fuc;

d.

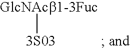

e. analogues and derivatives thereof

for the treatment of restenosis.

The invention further provides for the use of a purified extract from Microciona prolifera or a compound selected from the group consisting of:

a. GlcNAcβ1-3Fuc;

b. Galβ1-4GlcNAcβ1-3Fuc;

c. (4,6-Pyr)Galβ1-4GlcNAcβ1-3Fuc;

d.

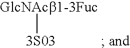

e. analogues and derivatives thereof

for the treatment of an autoimmune disease (such as multiple sclerosis, diabetes, and inflammatory joint disease (such as arthritis)).

The invention further provides for the use of a purified extract from Microciona prolifera or a compound selected from the group consisting of:

a. GlcNAcβ1-3Fuc;

b. Galβ1-4GlcNAcβ1-3Fuc;

c. (4,6-Pyr)Galβ1-4GlcNAcβ1-3Fuc;

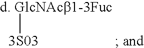

e. analogues and derivatives thereof

for the treatment of multiple sclerosis.

According to a further aspect of the present invention there is provided the use of an antibody which binds to a polypeptide comprising an amino acid sequence selected from the group consisting of B-1 (SEQ ID NO: 1), B-2 (SEQ ID NO: 2), B-3 (SEQ ID NO 3), B-6 (SEQ ID NO 4), B-9 (SEQ ID NO: 5), B-10 (SEQ ID NO: 6), B-11 (SEQ ID NO: 7) and analogues and derivatives thereof for the treatment of restenosis.

According to a further aspect of the present invention there is provided the use of an antibody which binds to a polypeptide comprising an amino acid sequence selected from the group consisting of B-1 (SEQ ID NO: 1), B-2 (SEQ ID NO: 2), B-3 (SEQ ID NO 3), B-6 (SEQ ID NO 4), B-9 (SEQ ID NO: 5), B-10 (SEQ ID NO: 6), B-11 (SEQ ID NO: 7) and analogues and derivatives thereof for the treatment of an autoimmune disease (such as multiple sclerosis, diabetes, and inflammatory joint disease (such as arthritis)).

According to a further aspect of the present invention there is provided the use of an antibody which binds to a polypeptide comprising an amino acid sequence selected from the group consisting of B-1 (SEQ ID NO: 1), B-2 (SEQ ID NO: 2), B-3 (SEQ ID NO 3), B-6 (SEQ ID NO 4), B-9 (SEQ D NO: 5), B-10 (SEQ ID NO: 6), B-11 (SEQ ID NO: 7) and analogues and derivatives thereof for the treatment of multiple sclerosis.

According to a further aspect of the invention there is provided a pharmaceutical composition for the treatment of restenosis comprising: a purified extract from Microciona prolifera having a peak at about 2.7 kDa using MALDI spectroscopy; and a pharmaceutically acceptable carrier.

According to a further aspect of the invention there is provided a pharmaceutical composition for the treatment of an autoimmune disease (such as multiple sclerosis, diabetes, and inflammatory joint disease (such as arthritis)) comprising: a purified extract from Microciona prolifera having a peak at about 2.7 kDa using MALDI spectroscopy; and a pharmaceutically acceptable carrier.

According to yet another aspect of the present invention, there is provided a pharmaceutical composition for the treatment of restenosis comprising: an extract from Microciona prolifera obtained by collecting cuttings from the tips of branches of Microciona prolifera;

a. washing the cuttings in cold calcium and magnesium free artificial seawater (CMFASW), wherein said cuttings contain one or more MAF-containing cells and a fibro-silacaceous support;

b. rotating the cuttings in CMFASW;

c. separating MAF-containing cells from the fibro-silacaceous support;

d. incubating and rotating the MAF-containing cells;

e. centrifuging the MAF-containing cells and retaining the supernatant;

f. adding EDTA and optionally a preservative to obtain a supernatant solution;

g. storing the supernatant solution at about 4 degrees for at least 120 hours;

h. dialyzing the supernatant to obtain the retenate; and

i. lyophilizing the retentate to obtain the extract.

According to yet another aspect of the present invention, there is provided a pharmaceutical composition for the treatment of an autoimmune disease (such as multiple sclerosis, diabetes, and inflammatory joint disease (such as arthritis)) comprising: an extract from Microciona prolifera obtained by collecting cuttings from the tips of branches of Microciona prolifera;

a. washing the cuttings in cold calcium and magnesium free artificial seawater (CMFASW); wherein said cuttings contain one or more MAF-containing cells and a fibro-silacaceous support;

b. rotating the cuttings in CMFASW;

c. separating MAF-containing cells from their fibro-silacaceous support;

d. incubating and rotating the MAF-containing cells;

e. centrifuging the MAF-containing cells and retaining the supernatant;

f. adding EDTA and optionally a preservative to obtain a supernatant solution;

g. storing the supernatant solution at about 4 degrees for at least 120 hours;

h. dialyzing the supernatant solution to obtain a retenate; and

i. lyophilizing the retentate to obtain the extract.

According to a further aspect of the present invention there is provided the use of a polypeptide comprising an amino acid sequence selected from the group consisting of B-1 (SEQ ID NO: 1), B-2 (SEQ ID NO: 2 ), B-3 (SEQ ID NO 3), B-6 (SEQ ID NO 4), B-9 (SEQ ID NO: 5), B-10 (SEQ ID NO: 6), and B-11 (SEQ ID NO: 7), analogues and derivatives thereof, purified extracts of Microciona prolifera, and carbohydrate compounds selected from the group consisting of

a. GlcNAcβ1-3Fuc;

b. Galβ1-4GlcNAcβ1-3Fuc;

c. (4,6-Pyr)Galβ1-4GlcNAcβ1-3Fuc;

d.

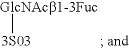

e. analogues and derivatives thereof,

to further identify ligands on cell surface that may serve as potential therapeutic targets.

According to yet another aspect of the present invention, there is provided a therapeutic targets comprising at least one ligand on a cell surface which bind to a compound selected from the group consisting of B-1 (SEQ ID NO: 1) B-2 (SEQ ID NO: 2 ) B-3 (SEQ ID NO 3), B-6 (SEQ ID NO 4), B-9 (SEQ ID NO: 5), B-10 (SEQ ID NO: 6), B-11 (SEQ ID NO: 7) and analogues and derivatives thereof, a purified extract from Microciona prolifera and a compound selected from the group consisting of

a. GlcNAcβ1-3Fuc;

b. Galβ1-4GlcNAcβ1-3Fuc;

c. (4,6-Pyr)Galβ1-4GlcNAcβ1-3Fuc;

d.

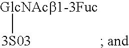

e. analogues and derivatives thereof.

### DESCRIPTION OF THE DRAWINGS

FIG. 1 shows photographs demonstrating the inhibition of control and peptide B-9-treated (20 μg/ml) smooth muscle cell motility at 0 and 16 hours.

FIG. 2 shows photographs demonstrating the inhibition of control and peptide B-2 -treated (50 μg/ml and 100 μg/ml) smooth muscle cell motility at 0 and 20 hours.

FIG. 3 shows photographs demonstrating the inhibition of control and carbohydrate RR-1-treated (1 μg/ml and 5 μg/ml) smooth muscle cell motility at 0 and 20 hours.

FIG. 4 shows photographs demonstrating the inhibition of control and Maf 2.7 kDa purified extract treated (10 μg/ml) smooth muscle cell motility at 0 and 20 hours.

FIG. 5 is a graph showing the effect of B-1 peptide on clinical scores in a mouse EAE model.

FIG. 6 is a graph showing the effect of B-9 peptide on clinical scores in a mouse EAE model.

FIG. 7 is a MALDI spectroscopy performed in negative polarities showing a predominant peak at 2.7 kDa.

FIG. 8 is a MALDI spectroscopy performed in positive polarities showing a predominant peak at 2.7 kDa

### DETAILED DESCRIPTION OF THE INVENTION

Definitions

“CFA” is complete Freunds' adjuvant.

“Combination therapies” means therapeutic agent combined with other therapeutic agent

“Compounds” mean agents for the treatment of diseases.

“Disease” means any abnormal condition of body functions or structure that is considered to be harmful to the affected individual and includes an illness or disorder

“EAE” means the experimental autoimmune encephalomyelitis mouse model; the mouse model for multiple sclerosis.

“MAF” is an adhesive proteoglycan from the marine sponge Microciona prolifera.

“MALDI” (Mass Analysis Matrix Assisted Laser Desorption)/Flight (MALDI-TOF) is a mass analysis technique

“MBP” is myelin basic protein.

“PBS” (Phosphate buffer saline) is an injectable solution that serves as a negative control because it does not have any physiological or therapeutic effects.

“PLP” is an isoprotein proteolipid protein. PLP becomes predominant in the adult.

“PTX ” (pertussis toxin) is the major protein toxin produced by virulent strains of Bordetella pertussis, the organism that causes whooping cough. Pertussis toxin (PTX) is a potent ancillary adjuvant that primes macrophages used to elicit several different autoimmune diseases, including experimental allergic encephalomyelitis (EAE)

Carbohydrates:

“RR1” is a non-sulfated disaccharide with the following structure:

GlcNAcβ1-3Fuc

“RR2” is a sulfated disaccharide with the following structure:

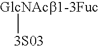

“RR4” is a non-pyruvated trisaccharide with the following structure:

Galβ1-4GlcNAcβ1-3Fuc

“RR5” is a pyruvated trisaccharide with the following structure:

(4,6-Pyr)Galβ1-4GlcNAcβ1-3Fuc

Peptides

“Peptide B-1” is a 13 amino acid synthetic peptide, representing the putative HA (hyaluronic acid) binding sequence of MAF (marine sponge Microciona prolifera adhesive proteoglycan), having the following amino acid sequence:

| | |
|---|---|
| SEQ ID NO: 1 | Gly-Val-Ser-Val-Arg-Arg-Tyr-Arg-Asn- |
| | Arg-Val-Arg-Ile |
| | (GVSVRRYRNRVRI) |

“Peptide B-2” is a 15 amino acid synthetic peptide, derived from MAF, having the following amino acid sequence:

| | |
|---|---|
| SEQ ID NO: 2 | Val-Gly-Phe-Asp-Pro-Tyr-Asp-Tyr-Glu- |
| | Val-Asn-Glu-Ala-Asp-Gly |
| | (VGFDPYDYEVNEADG) |

“Peptide B-3” is a 10 amino acid synthetic peptide derived from MAF having the following amino acid sequence:

| | |
|---|---|
| SEQ ID NO 3: | Glu-Asp-Gln-Leu-Asp-Ala-Met-Asn-Leu- |
| | Ser |
| | (EDQLDAMNLS) |

“Peptide B-6” is a 11 amino acid synthetic peptide derived from MAF having the following amino acid sequence:

| | |
|---|---|
| SEQ ID NO 4: | Gly-Ser-Gly-Ile-Gly-Asp-Glu-Pro-Thr- |
| | Thr-Ser |
| | (GSGIGDEPTTS) |

“Peptide B-9” is a 12 amino acid synthetic peptide derived from MAF having the following amino acid sequence:

| | |
|---|---|
| SEQ ID NO 5: | Arg-Phe-Val-Ile-Asn-Ile-Thr-Thr-Ser- |
| | Gly-Ser-Asp |
| | (RFVINITTSGSD) |

“Peptide B-10” is a 12 amino acid synthetic peptide derived from MAF having the following amino acid sequence:

| | |
|---|---|
| SEQ ID NO 6: | Cys-Phe-Leu-Thr-Pro-His-Gly-Val-Glu- |
| | Leu-His-Lys |
| | (CFLTPHGVELHK) |

“Peptide B-1”: is a 21 amino acid synthetic peptide derived from MAF having the following amino acid sequence:

| | |
|---|---|
| SEQ ID NO 7: | Asn-Gly-Ser-Ile-Gly-Pro-Arg-Gly-Leu- |
| | Pro-Gly-Val-Arg-Gly-Asp-Arg-Gly-Lys- |
| | Arg-Gly-Lys |
| | (NGSIGPRGLPGVRGDRGKRGK) |

The present invention is directed to novel peptides, carbohydrates and extracts that originate from the marine sponge Microciona prolifera and that have the ability to inhibit cell migration without affecting cell viability. These synthetic peptides are also effective in decreasing clinical signs of disease in the EAE mouse model for multiple sclerosis. Therefore, these peptides, carbohydrates and extracts have the potential to provide an effective treatment for restenosis, autoimmune, proliferative and viral diseases.

Peptides Originating from MAFp3

The present invention includes synthetic peptide constructs B-1 (SEQ ID NO: 1), B-2 (SEQ ID NO: 2), B-3 (SEQ ID NO 3), B-6 (SEQ ID NO 4), B-9 (SEQ ID NO: 5), B-10 (SEQ ID NO: 6), and B-11 (SEQ ID NO: 7) of a cloned peptide MAFp3 fragment (Fernandez-Busquets et al., 1996).

The peptides of the invention may be prepared by chemical synthesis using techniques well known in the chemistry of proteins such as solid phase synthesis (Merrifield et al., 1964) or synthesis in homogenous solution (Houbemweyl, 1987). The peptides of the invention may also be produced by recombinant DNA technology by the methods known in the art.

Antibodies may likewise be employed to treat or to prevent restenosis coronary intervention, autoimmune diseases, viral diseases and proliferative diseases. The invention also provides for antibodies which bind to polypeptides B-1 (SEQ ID NO: 1), B-2 (SEQ ID NO: 2 ), B-3 (SEQ ID NO 3), B-6 (SEQ ID NO 4), B-9 (SEQ ID NO: 5), B-10 (SEQ ID NO: 6), and B-11 (SEQ ID NO: 7). The antibodies are generated using methods well known to those in the art. One of ordinary skill in the art will appreciate that a variety of alternative techniques for generating antibodies exist. In this regard, the following U.S. patents teach a variety of these methodologies and are thus incorporated herein by reference: U.S. Pat. Nos. 5,840,479; 5,770,380; 5,204,244; 5,482,856; 5,849,288; 5,780,225; 5,395,750; 5,225,539; 5,110,833; 5,693,762; 5,693,761; 5,693,762; 5,698,435; and 5,328,834.

Once suitable antibodies have been obtained, they may be isolated or purified by many techniques well known to those of ordinary skill in the art (see Antibodies: A Laboratory Manual, Harlow and Lane (eds.), Cold Spring Harbor Laboratory Press, 1988). Suitable techniques include peptide or protein affinity columns, HPLC (e.g., reversed phase, size exclusion, ion-exchange), purification on protein A or protein G columns, or any combination of these techniques.

The invention also includes vaccines the polypeptides listed above. Vaccines can be made using any method known in the art.

Carbohydrates Derived from Marine Sponge M. prolifera

Carbohydrates of the present invention include:

GlcNAcβ1-3Fuc (RR-1);

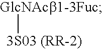

Galβ1-4GLcNAcβ1-3Fuc (RR-4); and

(4,6-Pyr)Galβ1-4GlcNAcβ1-3Fuc (RR-5). These carbohydrates can be prepared as described in Spillmann et al., 1995.

Preparation of Active Extracts from Microciona prolifera

Purified extracts of Microciona prolifera, the red bread marine sponge, may be obtained through any method known in the art. Preferably, live specimens of Microciona prolifera are collected. 1-2 mm cuttings from tips (growing portions) of branches on the whole sponge are removed, forming batches of approximately 300-500 grams. Several hundred branches may be present on a single adult sponge, thereby facilitating the collection of large quantities of cell-rich fragments. The cut pieces are first washed three times with cold calcium and magnesium free artificial seawater (CMFASW) prepared according to the MBL formulary, placed in a beaker with CMFASW and gently agitated on the rotating platform for four hours at 16° C. Aliquots of the soaked tips are then placed on CMF-washed silk bolting cloth which can be fashioned into a pouch in the gloved hand, and squeezed so as to express MAF-containing cells free of their fibro-silicaceous support into a second container. Next, in order to facilitate the release of intact MAF proteoglycan from MAF-containing cells, the suspension is then placed in a large Erlenmeyer flask and rotated at 120 rpm for six hours at 16° C.

The suspension is then centrifuged in the cold at 2500 rpm for 20 min to separate the MAF-containing supernatant. Cold centrifugation is repeated on the sponge cell-free supernatant fluid at a speed of 9000 RPM for 30 min so as remove fine particulate matter, such as bacteria, spores etc. Thereafter, EDTA is added at a concentration of 1 mM together with sodium azide at 1/2000 as a preservative. The batch of crude MAF extract is then maintained at 4° C. for at least 120 hr before any further processing. The combination of calcium depletion and EDTA chelation generates low molecular fragments from intact high molecular weight MAF whose carbohydrate subunits are linked by calcium.

Volume reduction may be performed by any method known to those skilled in the art. Preferably Spectra/Por dialysis tubing (Fisher catalog no. 08-671-26, 1000 MWCO) is heated in 1 mM EDTA, profusely rinsed with glass distilled water, and then are filled and embedded in polyethylene glycol (PEG-J, T. Baker catalog no. U 222-09) according to the following scheme. A shallow metal pan is lined with absorbent paper. The MAF-filled and clamped tubing is then placed in the pan in a snake-like configuration. Dry PEG powder is added so as to cover the tubing.

After the volume has been substantially reduced, preferably by 85-90%, the concentrated extract is centrifuged for 60 min at 18,600 rpm. The supernatant is then divided into two portions, each placed in suitably prepared Spectra/Por dialysis tubing (Fisher catalog no. 08-750-3B-1000 MWCO). Dialysis is then carried out against distilled water in the cold room. The dialysate water may be kept in motion by a stirring bar and a magnetic stirring apparatus. Preferably, the water is replaced daily for a period of six days in order to achieve a final diffusate/retentate ratio of 200. The retentate is then shell frozen and lyophilized.

The dry product may then be further purified prior to analysis. Membrane separation preferably takes place using Spectra/Por (Fisher catalog no. 08-750-SC-3500 MWCO). 0.6 g of dried product is dissolved in 10 ml glass distilled water and placed in washed dialysis tubing with a 3500 MWCO and anchored in a graduate cylinder containing 500 ml of water, and stored for 120 hr in the cold room. The diffusates are pooled, frozen on dry ice, and lyophilized.

Pharmaceutical Compositions

The present invention provides for pharmaceutical compositions comprising synthetic peptides, carbohydrates, as well as extracts from the marine sponge Microciona prolifera. Specifically, the pharmaceutical composition contains synthetic MAFp3 peptides B-2 (SEQ ID NO: 2 ), B-3(SEQ ID NO: 3), B-6(SEQ ID NO:4), B-9(SEQ ID NO: 5), B-10(SEQ ID NO:6), and B-11(SEQ ID NO: 7), carbohydrates RR-1, RR-2 , RR-4 and RR-5, their analogues, derivatives and conjugates and/or purified extracts from Microciona prolifera.

These compositions are useful for the treatment of a number of diseases, including restenosis, autoimmune, viral and proliferative diseases.

The pharmaceutical compositions of the invention preferably contain a pharmaceutically acceptable carrier or excipient suitable for rendering the compound or mixture administrable orally as a tablet, capsule or pill, or parenterally, intravenously, intradermally, intramuscularly or subcutaneously, rectally, via inhalation or via buccal administration, or transdermally. The active ingredients may be admixed or compounded with any conventional, pharmaceutically acceptable carrier or excipient. It will be understood by those skilled in the art that any mode of administration, vehicle or carrier conventionally employed and which is inert with respect to the active agents may be utilized for preparing and administering the pharmaceutical compositions of the present invention. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the invention, will experience no difficulty in determining suitable and appropriate vehicles, excipients and carriers or in compounding the active ingredients therewith to form the pharmaceutical compositions of the invention.

The compositions of the invention may also be conjugated to transport molecules, monoclonal antibodies or transport modalities such as vesicles and micelles that preferentially target recipient cells.

The composition of the present invention may also be formulated to be contained within, or, adapted to be released by a surgical or medical device or implant, such as, for example stents, sutures, catheters, prosthesis and the like Specifically, the active compound may be covalently linked or mixed or encapsulated in microcapsules with either polymeric or non-polymeric formulations which may coat, embed or impregnate or otherwise contact a medical device that is commercially available or is in research and development phase such as an implant, stent, stent graft, vascular graft, indwelling catheter, sutures, catheter, prosthesis and the like. In other cases, the active compound may contact a medical device such as an implant, stent, stent graft, vascular graft, indwelling catheter, sutures, catheter, prosthesis and the like without any formulations. Carriers can be either commercially available or in research and development phase.

The compounds of the present invention in the described dosages are administered orally, intraperitoneally, subcutaneously, intra-muscularly, transdermally, sublingually or intravenously as is known in the art. For example, for oral administration the pharmaceutical composition can be prepared orally, for example, in the form of tablets, troches, capsules, elixirs, suspensions, syrups, wafers, chewing gum or the like prepared by procedures known to those skilled in the art. The amount of active compound in such therapeutically useful compositions or preparations is such that a suitable dosage will be obtained.

The therapeutically effective amount of active agent to be included in the pharmaceutical composition of the invention depends, in each case, upon several factors, e.g., the type, size and condition of the patient to be treated, the intended mode of administration, the capacity of the patient to incorporate the intended dosage form, etc.

It can generally be stated that the peptide, carbohydrate and the principal active compound isolated from the extract, or analogues or derivatives thereof should be preferably administered in an amount of at least 0.1 μg/kg per injectable dose and up to 50 mg/kg per dose. In the case of a stent, the therapeutic amount of principal active compound to be administered to the intimal or lumenal layer of arterial walls may range from 0.01 μg to about 50mg.

### EXAMPLE 1

### Effect of Peptides Carbohydrates and Extract (2.7K) on Smooth Muscle Cell Migration—In Vitro Assay of Biological Activity

The following in vitro assays were conducted to illustrate the effect of peptides, carbohydrates and extract (2.7K) on the smooth muscle cell migration. The cells used in the following example were rat aortic smooth muscle cells, A-10. The cells were seeded at 2.5×10⁵ cells/well in 6 well plates using Dulbecco's Modified Eagle Medium (Gibco BRL Cat. # 11885-084),10% FBS and 1% Antibiotic Antimycotic (Gibco BRL Cat. # 15240). Confluency of cells after about 8 hrs was between 80˜90%. Cells were injured with the single edge cell scraper (one injury/well) then washed twice with PBS and treated with peptides B-1 (SEQ ID NO:1), B-2 (SEQ ID NO:2 ), B-3 (SEQ ID NO: 3), B-6, (SEQ ID NO: 4), B-9(SEQ ID NO:5), B-10(SEQ ID NO:6) AND B-11(SEQ ID NO: 7) at concentrations ranging from 10 μg/ml ml to 100 μg/ml. Images were taken using a X5 modulation objective (Zeiss, Germany) attached to a Zeiss Axiovert 100 inverted microscope equipped with Hoffman Modulation contrast optical filters (Greenvale, N.Y.).

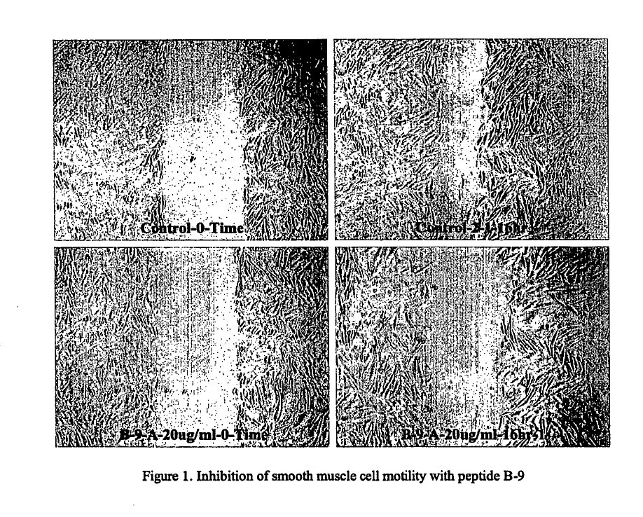

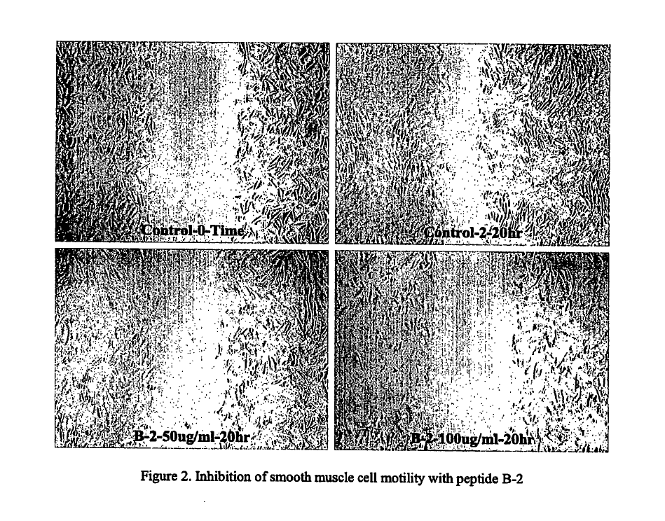

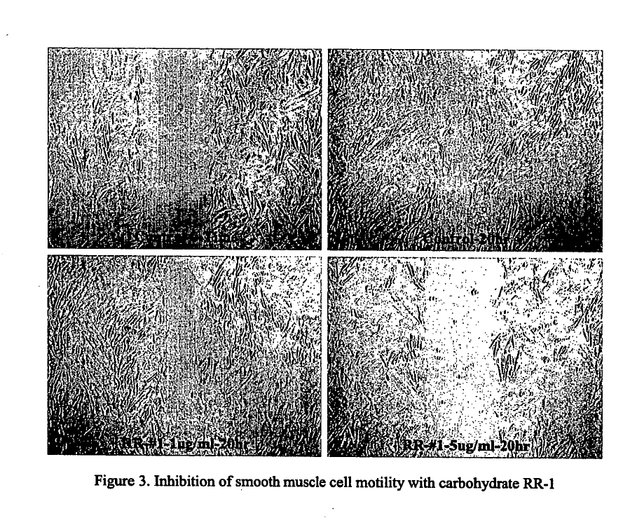

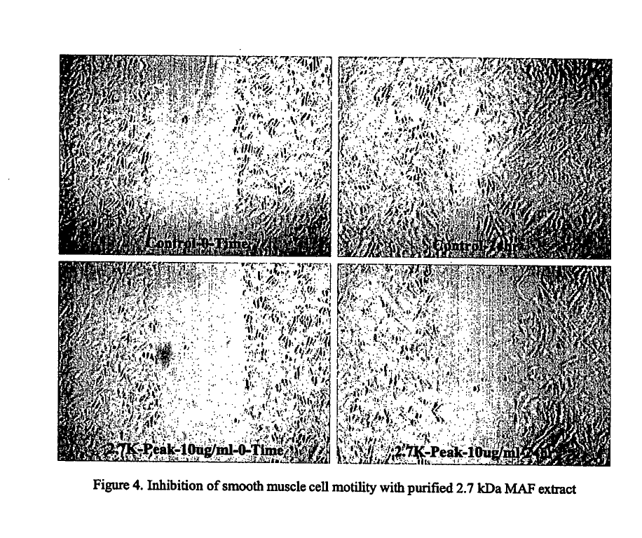

The images were taken at zero time and following the incubation of smooth muscle cells with peptide, carbohydrates and extracts for a period of up to 24 hrs (FIGS. 1, 2, 3 and 4). The 100% inhibition of cell motility without toxic effects was achieved with the peptide concentrations ranging from 10 μg/ml to 100 μg/ml, whereas the 100% inhibition with carbohydrates and 2.7K purified MAF extract was achieved with the concentrations ranging from 2 to 25 μg/ml. The 100% inhibition of cell motility data is summarized in Tables 1 and 2.

TABLE 1
Inhibition of smooth muscle cell motility with MAFp3 peptides

| MAFp3 | 100% Inhibition of Cell |
|---|---|
| Peptides | Motility |
| B-1 | 100 μg/ml |
| B-2 | 50-100 |
| B-3 | 50 |
| B-6 | 50˜75 |
| B-9 | 20 |
| B-10 | 100 |
| B-11 | 75˜100 |

TABLE 2
Inhibition of smooth cell motility with MAF
carbohydrates and MAF 2.7 K extract

| MAF | 100% Inhibition |
|---|---|
| Oligosaccharides | of Cell Motility |
| MAF-2.7 K | 2˜10 μg/mL |
| RR-1 disaccharides | 5˜10 |
| RR-5 trisaccharides | 25 |

The results of the assay demonstrate that the compounds of the invention are efficient inhibitors of smooth muscle cell migration in vitro and therefore believed that they may serve as an endogenous inhibitor of smooth muscle cell migration in the intimal lesion after injury as well as in prevention and treatment of proliferative, viral and autoimmune diseases. Increased smooth muscle cell migration following injury is one of the predominant causes of obstruction of the vascular system following stenting and other vascular procedures. Specifically, smooth muscle cell migration is a major cause of restenosis (For a review, see Schwartz, 1997, Raines, 2000).

### EXAMPLE 2

### Effect of B-1 and B-9 Peptides on EAE Mouse Model

The EAE mouse model is the standard animal model used for testing therapeutic compounds in the treatment of multiple sclerosis (MS). Acute EAE was induced by immunization of 3 months old SJL/J female mice (Jackson Lab.; Bar Harbor, Me.) with the MBP (myelin basic protein) and PTX (pertussis toxin). Each animal received an s.c. injection at tail base of 200 μg MBP in 0.1 ml of CFA and received an i.v. injection of 200 ng of PTX; pertussis toxin was injected again 48 hours later. Mice (4 animals per group) were treated with the peptide B-1 (SEQ ID NO: 1) or peptide B-9 (SEQ ID NO: 5) at a dose of 5 mg/kg daily; injections starting on the day of first immunization. Treatment was stopped at time of sacrifice. Mice were monitored daily from day 7 after immunization for clinical signs of EAE and were scored on a scale of 0 to 5. A score of 0 represented the absence of signs while a score of 5 was given to moribund animals.

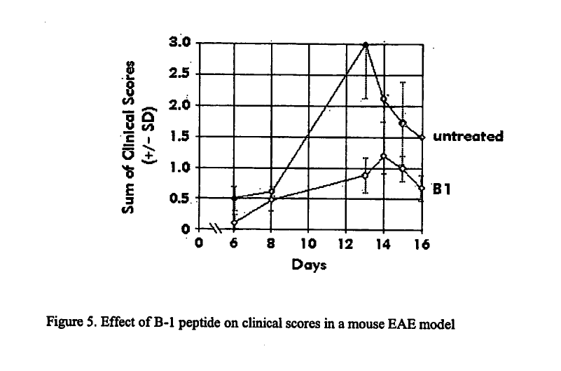

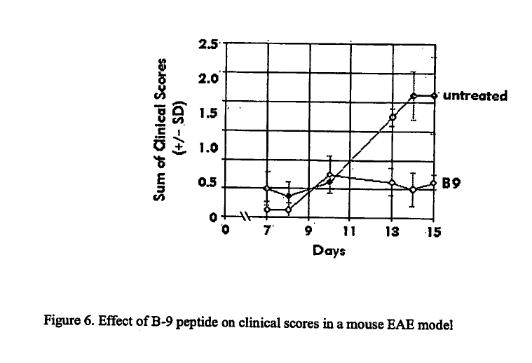

A marked improvement of mean clinical score was observed by day 13 at both groups treated with the peptides. At day 15, the mean clinical score was 1.7 in PBS treated mice compared to 0.6 in B-9 treated animals; whereas the mean clinical score was 1.5 in PBS treated mice compared to 0.7 in B-1 treated animals. Treatment of the EAE mice with B-1 and B-9 peptide showed significant attenuation of clinical signs of MS symptoms by improvement in mean clinical score and a delay in progression to disability. As shown in FIGS. 5 and 6, the peptide treatments were able to decrease the clinical scores up to 60% in comparison to non-treated animals.

### EXAMPLE 3

### Preparation of Active Extracts from Microciona prolifera

Live specimens of Microciona prolifera, the red bread marine sponge, were collected in the Cape Cod area by personnel of the Marine Resources Center (MRC) at the Marine Biological Laboratory (MBL), Woods Hole, Mass. The adhesive proteoglycan of sponge, MAF, was prepared from 1-2 mm cuttings from tips (growing portions) of branches on the whole sponge. Several hundred branches may be present on a single adult sponge, thereby facilitating the collection of large quantities of cell-rich fragments. A single batch was prepared from the cuttings derived from five adult sponges, each weighing 300-500 gm. The cut pieces were first washed three times with cold calcium and magnesium free artificial seawater (CMFASW) prepared according to the MBL formulary. They were placed in a beaker with CMFASW and were gently agitated on the rotating platform for four hours at 16° C. Aliquots of the soaked tips were then placed on CMF-washed silk bolting cloth which can be fashioned into a pouch in the gloved hand, and squeezed so as to express MAF-containing cells free of their fibro-silicaceous support into a second container. The yield of cells was ca.2-2.5×10⁷ cells per ml. The suspension was then placed in a large Erlenmeyer flask and rotated at 120 rpm for six hours at 16° C., a process, which facilitates the release of intact MAF proteoglycan from MAF-containing cells.

Cells were separated from the MAF-containing supernatant by centrifuging the suspension in the cold at 2500 rpm for 20 min. Cold centrifugation was repeated on the sponge cell-free supernatant fluid at a speed of 9000 RPM for 30 min so as remove fine particulate matter, such as bacteria, spores etc. Thereafter, EDTA was added at a concentration of 1 mM together with sodium azide at 1/2000 as a preservative. The batch of crude MAF extract, consisting of 2-2.5 liters was maintained at 4° C. for at least 120 hr before any further processing. The combination of calcium depletion and EDTA chelation generates low molecular fragments from intact high molecular weight MAF whose carbohydrate subunits are linked by calcium.

Volume reduction was carried out in Spectra /Por dialysis tubing (Fisher catalog no. 08-671-26, 1000 MWCO) which had been heated in 1 mM EDTA beforehand and profusely rinsed with glass distilled water. The filled dialysis tubes were embedded in polyethylene glycol (PEG-J, T. Baker catalog no. U 222-09) according to the following scheme. A shallow metal pan was lined with absorbent paper. The MAF-filled and clamped tubing was then placed in the pan in a snake-like configuration. Dry PEG powder was then added so as to cover the tubing. At 48 hr an 85-90% reduction in volume had occurred. The concentrated brown-colored extract was spun for 60 min at 18,600 rpm, a procedure that yielded a dark brown pellet, and a substantial reduction of color in the supernatant. This was divided into two portions, each placed in suitably prepared Spectra/Por dialysis tubing (Fisher catalog no. 08-750-3B-1000 MWCO). Dialysis was carried out against distilled water in the cold room. The dialysate water was kept in motion by a stirring bar and a magnetic stirring apparatus. The water was replaced daily for a period of six days to achieve a final diffusate/retentate ratio of 200. The retentate was shell frozen and lyophilized.

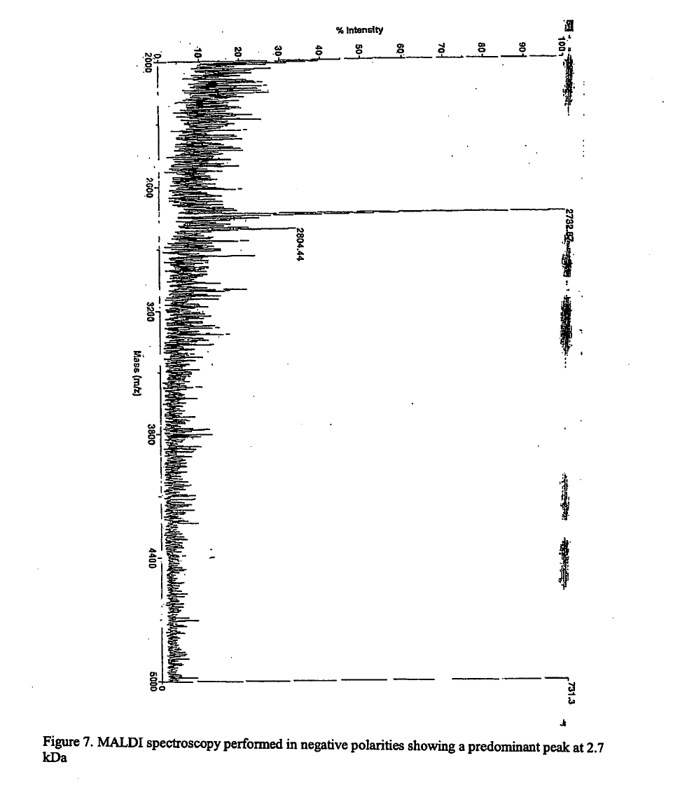

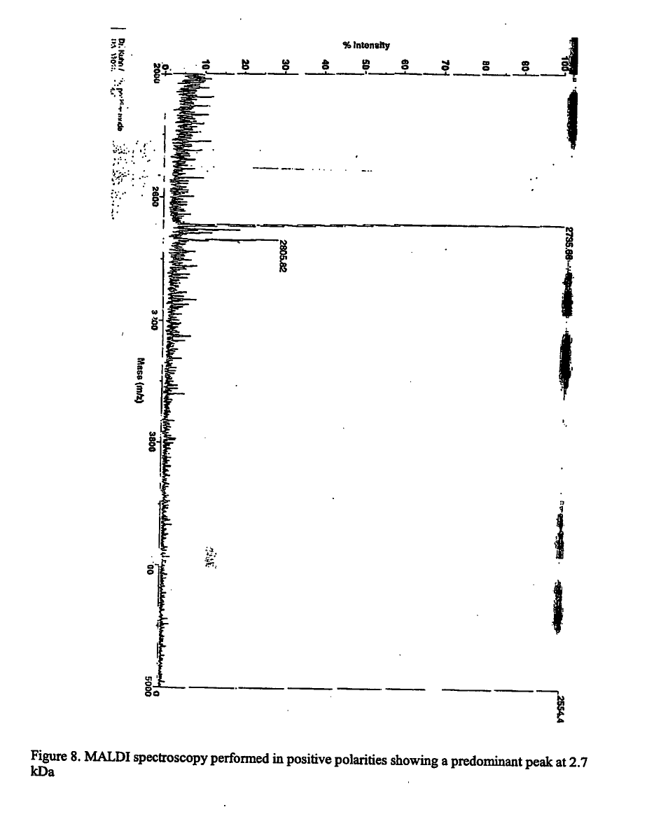

Membrane separation using Spectra/Por (Fisher catalog no. 08-750-5C -3500 MWCO). 0.6 g of dried product dissolved in 10 ml glass distilled water was placed in washed dialysis tubing with a 3500 MWCO and anchored in a graduate cylinder containing 500 ml of water, and stored for 120 hr in the cold room. The diffusates were pooled, frozen on dry ice, and lyophilized. On examination of this material using MALDI spectroscopy a predominant peak was detected at 2.7 kDa. The result was consistent when MALDI spectroscopy was performed in negative and positive polarities (FIG. 7 and FIG. 8).

Although the invention has been described with preferred embodiments, it is to be understood that modifications may be resorted to as will be apparent to those skilled in the art. Such modifications and variations are to be considered within the purview and scope of the present invention.

References

Fernandez-Busquets X, Kammerer R A and Burger M M. A 35 kDa Protein Is the Basic Unit of the Core from the 2×10 4-kDa. Aggregation Factor Responsible for Species-specific Cell Adhesion in the Marine Sponge Microciona prolifera. 1996. Journal of Biological Chemistry 271(38): 23558-23565

Haseley S R, Vermeer H J, Kamerling J P, Vliegenthart J F. Carbohydrate self-recognition mediates marine sponge cellular adhesion. 2001. Proceedings of National Academy of Sciences 98(16): 9419-9424

Houbemweyl, Methods of organic chemistry, ed. E. Wansch, Vol. 15 I and II, Tricme, Stuttgart, 1987.

Jarchow J, Fritz J, Anselmetti D, Calabro A, Hascall V C, Gerosa D, Burger M M, Fernandez-Busquets X. Supramolecular Structure of a New Family of Circular Proteoglycans Mediating Cell Adhesion in Sponge. 2000. Journal of Structural Biology 132: 95-105

Merrifield, Journal of American Chemistry Association 85: 2149-2154, 1964

Misevic G N, Finne J, Burger M M. Involvement of carbohydrates as multiple low affinity interaction sites in the self-association of the aggregation factor from the marine sponge Microciona prolifera. 1987. Journal of Biological Chemistry 262(12 ):5870-7

Raines, E W. The extracellular matrix can regulate vascular cell migration, proliferation and survival. 2000. International Journal of Experimental Pathology 81:173-182

Schwartz, S M. Smooth Muscle Migration in Atherosclerosis and Restenosis. 1997. Journal of Clinical Investigation 99(12): 2814-2817

Spillmann D, Thomas-Oates J E, van Kuik J A, Viegenthart J F, Misevic G, Burger M M, Finne J. Characterization of a Novel Sulfated Carbohydrate Unit Implicated in the Carbohydrate-Carbohydrate-mediated Cell Aggregation on the Marine Sponge Microciona prolifera. 1995. Journal of Biological Chemistry 270(10): 5089-5097

1. A polypeptide comprising an amino acid sequence selected from a group consisting of B-2 (SEQ ID NO: 2 ), B-3 (SEQ ID NO 3), B-6 (SEQ ID NO 4), B-9 (SEQ ID NO: 5), B-10 (SEQ ID NO: 6), and B-11 (SEQ ID NO: 7).

2. A pharmaceutical composition comprising a at least one polypeptide of claim 1 or an analogue or derivative thereof.

3. A The pharmaceutical composition of claim 2 for the treatment of restenosis or an autoimmune disease.

4. (Canceled)

5. The pharmaceutical composition of claim 3, wherein the autoimmune disease is selected from the group consisting of multiple sclerosis, diabetes, inflammatory joint disease, and arthritis.

6-9. (Canceled)

10. An antibody which binds to a polypeptide comprising an amino acid sequence selected from a group consisting of B-1 (SEQ ID NO: 1), B-2 (SEQ ID NO: 2), B-3 (SEQ ID NO 3), B-6 (SEQ ID NO 4), B-9 (SEQ ID NO: 5), B-10 (SEQ ID NO: 6), B-11 (SEQ ID NO: 7), and analogues and derivatives thereof.

11. An antibody according to claim 10, wherein the antibody is a human monoclonal antibody.

12. An antibody according to claim 10, wherein the antibody is a Fab fragment of an antibody.

13. A vaccine composition for the treatment of a disease selected from a group consisting of autoimmune diseases and restenosis comprising an antigen for the antibody of claim 10.

14. A method for treating restenosis or an autoimmune disease comprising a step of administering to a patient at least one compound selected from a group consisting of (a) a polypeptide comprising an amino acid sequence selected from a group consisting of B-1 (SEQ ID NO: 1), B-2 (SEQ ID NO: 2), B-3 (SEQ ID NO 3), B-6 (SEQ ID NO 4), B-9 (SEQ ID NO: 5), B-10 (SEQ ID NO: 6), B-11 (SEQ ID NO: 7), and analogues and derivatives thereof; and (b) an antibody to the polypeptide of (a).

15. (Canceled)

16. The method of claim 14 wherein the autoimmune disease is selected from the group consisting of multiple sclerosis, diabetes, inflammatory joint disease and arthritis.

17-20. (Canceled)

21. A method for treating restenosis, an autoimmune disease, multiple sclerosis, diabetes, inflammatory joint disease or arthritis comprising a step of administering to a patient at least one compound selected from a group consisting of:
a. GlcNAcβ1-3Fuc;
b. Galβ1-4GlcNAcβ1-3Fuc;
c. (4,6-Pyr)Galβ1-4GlcNAcβ1-3Fuc:
d. 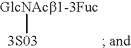
e. analogues and derivatives thereof.

22-27. (Canceled)

28. A method for treating restenosis or an autoimmune disease comprising a step of administering to a patient a pharmaceutical composition comprising a purified extract from Microciona prolifera.

29. (Canceled)

30. The method of claim 29 wherein the autoimmune disease selected from the group consisting of multiple sclerosis, diabetes, inflammatory joint disease and arthritis.

31-40. (Canceled)

41. The method according to claim 14, wherein a dose is administered according to a regime selected from a group consisting of a single dose, multiple daily doses, multiple weekly doses and multiple monthly doses.

42-61. (Canceled)

62. A pharmaceutical composition comprising:
a. a purified extract from Microciona prolifera having a peak at about 2.7 kDa using MALDI spectroscopy; and
b. a pharmaceutically acceptable carrier.

63. The pharmaceutical composition of claim 64 for the treatment of restenosis or an autoimmune disease.

64. The pharmaceutical composition of claim 63, wherein the autoimmune disease is selected from a group consisting of multiple sclerosis, diabetes, an inflammatory joint disease, and arthritis.

65. (Canceled)

66. A pharmaceutical composition for the treatment of restenosis, or an autoimmune disease comprising, an extract from Microciona prolifera obtained by
a. washing one or more cuttings of Microciona prolifera in a solution of cold calcium and magnesium free artificial seawater (CMFASW), wherein said cuttings contain one or more MAF-containing cells and a fibro-silacaceous support;
b. rotating the cuttings in CMFASW;
c. separating the MAF-containing cells from the fibro-silacaceous support;
d. incubating and rotating the MAF-containing cells;
e. centrifuging the MAF-containing cells and retaining the supernatant;
f. adding EDTA and optionally a preservative to obtain a supernatant solution;
g. storing the supernatant solution at about 4 degrees for at least 120 hours;
h. dialyzing the supernatant solution to obtain a retenate; and
i. lyophilizing the retenate to obtain the extract.

67. (Canceled)

68. The pharmaceutical composition of claim 67 wherein the autoimmune disease is selected from the group consisting of multiple sclerosis, diabetes, an inflammatory joint disease, and arthritis.

69-76. (Canceled)
